(19) United States
(12) Patent Application Publication
(10) Pub. No.: US 2013/0310805 A1
(43) Pub. Date: Nov. 21, 2013

(54) DELIVERY OF SUBSTANCES TO THE EXTERNAL AUDITORY CANAL AND NASAL CAVITY

(71) Applicant: OTITOPIC INC., Los Angeles, CA (US)

(72) Inventor: Kambiz YADIDI, Los Angeles, CA (US)

(73) Assignee: OTITOPIC INC., Los Angeles, CA (US)

(21) Appl. No.: 13/843,193

(22) Filed: Mar. 15, 2013

Related U.S. Application Data
(60) Provisional application No. 61/649,800, filed on May 21, 2012.
(60) Provisional application No. 61/707,291, filed on Sep. 28, 2012.

(51) Int. Cl. A61M 11/00; A61M 13/00

(52) U.S. Cl. CPC A61M 11/00; A61M 13/00
USPC 604/514; 604/58

(57) ABSTRACT

A device for delivery of a substance to an external auditory canal and/or nasal cavity can include a reservoir, an aerosolizing mechanism, a nozzle, and a retaining member. The reservoir can contain the substance. The aerosolizing mechanism can suspend a dose of the substance, as particulates for example, in a gas. The nozzle can include a lumen, an outer surface, and a distal end. The lumen can be sized to permit movement of the gas and particulates therethrough. The retaining member can be configured to be attached to the nozzle proximate the distal end of the nozzle, projecting laterally from the lumen of the nozzle, shaped to engage the ear and/or nostril, and can include a passage therethrough to allow passage of gas and to inhibit passage of at least some of the particulates.

### CROSS-REFERENCE TO RELATED APPLICATIONS

This application claims the benefit of U.S. Provisional Application No. 61/649,800, filed May 21, 2012, and U.S. Provisional Application No. 61/707,291, filed Sep. 28, 2012, the entirety of each of which is incorporated herein by reference.

### FIELD

The subject technology relates generally to apparatuses and methods for delivery of substances, e.g., medication, to the external auditory canal and the nasal cavity.

### BACKGROUND

A variety of conditions may afflict the external auditory canal. For example, the external auditory canal may become inflamed by infection or other malady, e.g., cellulitis. Also, a variety of conditions may be treated by delivery of substances to the nasal cavity.

### SUMMARY

An aspect of at least one embodiment disclosed herein includes the recognition of a need for improved apparatuses and methods for delivery of substances, such as medications, to the external auditory canal, i.e., the external ear canal. Some embodiments disclosed herein can delivery substances to other bodily locations, including the nose and mouth for example, in addition or alternative to the external auditory canal. Some embodiments can be particularly useful for delivery of dry substances, e.g., medications, to the external auditory canal while inhibiting the escape of particulates therefrom. Some embodiments can retain dry, wet, solid, liquid, or other particulates within the external auditory canal. In some embodiments, the same apparatus can be used to deliver substances both the external auditory canal and the nasal cavity.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 55. The other clauses can be presented in a similar manner.

1. A device for delivery of particulates to an external auditory canal, the device comprising:
a reservoir configured to contain a substance to be delivered to the external auditory canal;
an aerosolizing mechanism configured to suspend a dose of the substance, as particulates, in a gas;
a nozzle comprising (i) a lumen sized to permit movement of the suspended particulates therethrough, (ii) an outer surface, and (iii) a distal end;
a retaining member configured to be attached to the nozzle proximate the distal end of the nozzle, projecting laterally from the lumen of the nozzle, shaped to engage an external portion of the ear, and comprising a passage therethrough configured to allow passage of air and to inhibit passage of at least some of the particulates.

2. The device of Clause 1, wherein the particulates are not liquid.

3. The device of Clause 1, wherein the retaining member is spaced from the distal end of the nozzle.

4. The device of Clause 1, wherein the retaining member completely surrounds a circumference of the nozzle.

5. The device of Clause 1, further configured such that a volumetric flow rate through the nozzle and a volumetric flow rate through the retaining member are substantially the same during delivery.

6. The device of Clause 1, wherein at least some of the one or more passages of the retaining device have a characterizing dimension that is smaller than a particulate dimension.

7. The device of Clause 1, wherein the one or more passage of the retaining device are formed by an open-cell foam.

8. The device of Clause 1, wherein the one or more passage of the retaining device are formed by a mesh.

9. The device of Clause 1, wherein the retaining member further comprises a resilient portion configured to conform to a shape of the external ear and wherein the one or more passages of the retaining member are surrounded by one or more rigid walls.

10. The device of Clause 1, wherein the retaining member impedes advancement of the nozzle into the ear.

11. The device of Clause 10, wherein the retaining member has a portion with an outer dimension that is significantly larger than an opening to the external auditory canal.

12. The device of Clause 11, wherein the portion of the retaining member is at a distal end of the retaining member.

13. The device of Clause 1, wherein the retaining member is configured to be selectively attached and removed from the nozzle.

14. The device of Clause 1, wherein the reservoir is configured to contain a single dose of the substance.

15. The device of Clause 1, further comprising a desiccant in communication with the substance in the reservoir.

16. The device of Clause 1, wherein the aerosolizing mechanism is configured to suspend the particulates in the gas by one or more of a jet, an ultrasonic wave, or a vibrating mesh.

17. The device of Clause 16, wherein the aerosolizing mechanism comprises a bellows.

18. The device of Clause 16, wherein the aerosolizing mechanism comprises a compressed air unit.

19. The device of Clause 1, further comprising a mechanism for de-aggregating the substance into particulates.

20. The device of Clause 1, wherein the retaining member comprises a plurality of passages therethrough configured to allow passage of air and to inhibit passage of at least some of the particulates.

21. The device of Clause 1, wherein the substance to be delivered comprises one or more ingredients selected from the group consisting of antibiotics, antibacterial agents, bacteriocides, bacteriostatic agents, antifungals, antivirals, analgesics, anesthetics, anti-inflammatories, nonsteroidal anti-inflammatory drugs, immunomodulators, tacrolimus, pimecrolimus, cerumenolytics, Debrox, carbamide peroxide, glycerine, sodium bicarbonate, Cerumol, Cerumenex, Exterol, and Docusate.

22. A device for impeding escape of particulate material from an external auditory canal during delivery, comprising a retaining member configured to be attached to a nozzle proximate a distal end of the nozzle and to engage an external portion of the ear, the retaining member comprising one or more passages therethrough configured to allow passage of air and to inhibit passage of at least some of the particulates.

23. The device of Clause 22, further comprising a nozzle having a lumen sized to permit movement of suspended particulates therethrough, the retaining member being attached to the nozzle.

24. The device of Clause 23, wherein the retaining member and the nozzle are integrally formed.

25. A method for delivering particulate material to an external auditory canal, comprising:
aerosolizing a dose of a substance;
delivering the substance to the external auditory canal through a nozzle; and
inhibiting egress of at least some of the substance from the external auditory canal between the nozzle and a wall of the external auditory canal.

26. The method of Clause 25, wherein said inhibiting comprises reducing an amount of cross-sectional area between the nozzle and the external auditory canal through which air and the substance can escape the external auditory canal.

27. The method of Clause 26, wherein said inhibiting further comprises spanning an opening of the external auditory canal with a retaining member attached to the nozzle proximate the distal end of the nozzle and comprising one or more passages therethrough configured to allow passage of air and to inhibit passage of at least some of the particulates.

28. The method of Clause 25, wherein the substance comprises one or more ingredients selected from the group consisting of antibiotics, antibacterial agents, bacteriocides, bacteriostatic agents, antifungals, antivirals, analgesics, anesthetics, anti-inflammatories, nonsteroidal anti-inflammatory drugs, immunomodulators, tacrolimus, pimecrolimus, cerumenolytics, Debrox, carbamide peroxide, glycerine, sodium bicarbonate, Cerumol, Cerumenex, Exterol, and Docusate.

29. The method of Clause 26, wherein the substance is delivered to treat an external auditory canal infection.

30. The method of Clause 26, wherein the substance is delivered to treat an abscess.

31. A device for delivery of particulates to an external auditory canal and a nasal cavity, comprising:
a reservoir configured to contain a substance to be delivered;
an aerosolizing mechanism configured to suspend a dose of the substance, as particulates, in a gas;
a nozzle comprising (i) a lumen sized to permit movement of the suspended particulates therethrough, (ii) an outer surface, and (iii) a distal end;
a retaining member configured to be attached to the nozzle proximate the distal end of the nozzle, projecting laterally from the lumen of the nozzle, shaped to engage independently each of (i) an external portion of the ear and (2) a nostril, and comprising a passage therethrough configured to allow passage of air and to inhibit passage of at least some of the particulates.

32. A device for impeding escape of particulate material from an external auditory canal or a nostril during delivery, comprising a retaining member configured to be attached to a nozzle proximate a distal end of the nozzle and to engage independently (i) an external portion of the ear and (ii) the nostril, the retaining member comprising one or more passages therethrough configured to allow passage of air and to inhibit passage of at least some of the particulates.

33. A method for delivering particulate material to a nasal cavity, comprising:
aerosolizing a dose of a substance;
delivering the substance to the nasal cavity through a nozzle; and
inhibiting egress of at least some of the substance from the nasal cavity between the nozzle and a wall of a nostril.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is a schematic view of an ear in partial cross section and an apparatus for delivering a substance to an external auditory canal according to an embodiment.

FIG. 2 is a cross-sectional view of an embodiment of a nozzle and a retaining member according to an embodiment.

FIG. 3 is a front view of the nozzle and retaining member of FIG. 2.

FIG. 4 is a cross-sectional view of an embodiment of a nozzle and a retaining member according to an embodiment.

FIG. 5 is a cross-sectional view of the nozzle and retaining member of FIG. 4 taken along line 5-5, shown in FIG. 4.

FIG. 6 is a cross-sectional view of an embodiment of a nozzle and a retaining member according to an embodiment.

FIG. 7 is a rear view of the nozzle and retaining member of FIG. 6.

FIG. 8 is a cross-sectional view of a nozzle and a retaining member according to an embodiment of the present invention.

FIG. 9 is a rear view of the nozzle and retaining member of FIG. 8.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

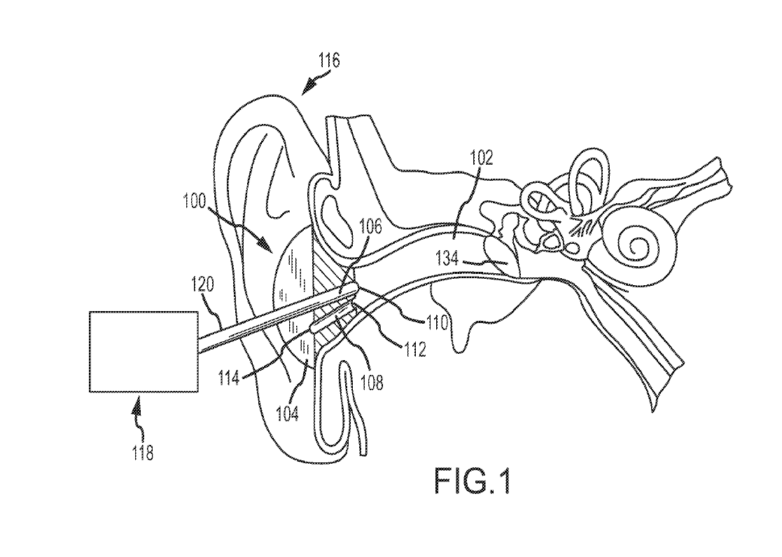

FIG. 1 is a schematic illustration of an ear and an embodiment of a device 100 for delivering a substance to an external auditory canal 102 and/or a nasal cavity (not shown). The substance to be delivered can be a solid or a liquid. In some embodiments, the solid or liquid substance to be delivered is a medication. In some embodiments, a medication can be delivered as a dry power. Non-limiting examples of medications that can be delivered using the devices and methods of the subject technology include antibiotics, antibacterial agents, bacteriocides, bacteriostatic agents, antifungals, antivirals, analgesics, anesthetics, anti-inflammatories, nonsteroidal anti-inflammatory drugs (NSAIDs), immunomodulators, tacrolimus, pimecrolimus, cerumenolytics, Debrox, carbamide peroxide, glycerine, sodium bicarbonate, Cerumol, Cerumenex, Exterol, and Docusate. Any of the aforementioned compounds can be delivered as a sole active ingredient, or in combination with other active ingredients, such as those identified herein and any other that may be desirable to be delivered. Non-limiting examples of combinations that can be delivered using the subject technology include a combination of carbamide peroxide (e.g., 6.5%) and glycerine; a combination of sodium bicarbonate and glycerine; a combination of arachis oil, turpentine and dichlorobenzene; a combination of Triethanolamine, polypeptides and oleate-condensate; and a combination of urea hydrogen peroxide and glycerine. The substance(s) to be delivered, including some or all of the above-referenced medications, can be converted to a powder from a gel, liquid, or solid by technologies known to those of skill in the art. For example, lyophilization is used in some embodiments the preparation of a dry powder for delivery.

One or more medications can be delivered using the subject technology to treat conditions of the external auditory canal, e.g., external auditory canal infections and abscesses, and nasal cavity, e.g., sinus infections.

As illustrated in FIG. 1, the delivery device 100 can comprise a retaining member 104 and a dispenser 118 configured to be connected for delivery of a substance. The retaining member 104 and the dispenser 118 can be connected directly or by an intervening structure, such as a tube 120, for example.

The retaining member 104 can comprise a delivery passage 106 and an outlet passage 108 connecting the external auditory canal with the outside environment. At least one delivery passage 106 can have a size sufficient to permit delivery of a dose of the substance through the retaining member 104 to the external auditory canal. The delivery passage 106 can comprise an opening 110 at its distil end for disbursement of the substance to the external auditory canal. At least one outlet passage 108 can have a size sufficient to permit air to escape from the external auditory canal during delivery of the substance while inhibiting escape of the substance from the external auditory canal. The outlet passage 108 can comprise a distal opening 112 and a proximal opening 114.

In some embodiments, the retaining member 104 can be configured to deliver a substance to the external auditory canal with little or no discomfort to the patient and without damaging the ear, e.g. the ear drum 134. In some embodiments, the delivery passages 106 and outlet passages 108 can be configured such that a rate of delivery of a stream, spray, or other disbursement of particles to the external auditory canal 102 through the delivery passage 106, and a resistance to flow through the outlet passages 108 do not produce an undesirable increase in localized or general pressure within the external auditory canal.

The delivery passages 106 can be configured such that, considering an output of the dispenser 118, the delivery rate of the substance and air is slow enough to avoid or reduce the risk of injury to the ear, yet sufficiently fast to promote adequate dispersion to the external auditory canal. The outlet passages 108 can be sufficiently large, numerous, or both that the retaining member 104, when placed against the ear for delivery, as to allow escape of air from the external auditory canal at a rate sufficient to prevent a rapid increase in pressure within the external auditory canal under normal circumstances.

The device 100 can be sufficiently long to extend generally from the outer ear 116 into the external auditory canal 102. In some embodiments, the delivery device 100 can contact the outer ear 116 to position the opening 110 of the delivery passage 106 for delivery of substance to the external auditory canal 102 without extending a substantial distance therein. In some embodiments, the delivery device 100 can extend into the external auditory canal 102 without substantially contacting the outer ear 116. In some embodiments, contact between the device 100 and the outer ear 116 can facilitate proper placement of the delivery device 100 for the comfort and safety of the ear.

The dispenser 118 can mix particles of a substance with a gas and propel the mixture of gas and particles for delivery. For example, the dispenser 118 can be an atomizer, a nebulizer, or other aerosolizing mechanism, including those now known and those that may be later developed, such as dispensers that operate by a jet, an ultrasonic wave, or a vibrating mesh. The dispenser 118 can be actuated by hand or other mechanism. In one embodiment, a dispenser can comprise a hand-driven bellows. In some embodiments, compressed air can be employed to delivery the substance in a gas. In some embodiments, the dispenser 118 can be configured to de-aggregate a substance into fine particles for delivery. Dispensers can suspend particles of the substance in a gas in some embodiments.

The dispenser 118 can optionally include a reservoir, container, or other storage device for containing a substance, e.g., medication, to be delivered. In some embodiments, the storage device can be a reservoir that is removably attachable to the dispenser 118 for refilling or replacement with another reservoir containing substance to be delivered.

The storage device of the dispenser 118 can contain sufficient substance for delivery of a plurality of doses or can comprise a plurality of metered doses packaged separately, for example in separate blisters. The dispenser 118 can be configured to deliver a meter dose of a substance de-aggregated from a larger quantity in some embodiments.

Storage of the substance to be delivered can be stored in a reservoir that is replaceable, such as a cartridge, or refillable. In some embodiments, the provision of a dose in a pre-metered dosage in a blister or a cartridge can facilitate delivery of an accurate dose of medication. Where multiple doses of medication or other substance are stored together in a common space, the dispenser 118 can be configured to separate a metered dose of the substance from a larger quantity held in the space, such as upon dispensing of a dose of the substance. In some embodiments, the storage mechanism can comprise a desiccant to preserve the medication or other substance. In some embodiments, the desiccant can comprise calcium carbonate or activated charcoal for example.

In some embodiments, a delivery passage 106 of the retaining member 104 can be formed by a nozzle. The nozzle can form the delivery passage 106 such that the delivery passage 106 has a constant cross-sectional area, e.g., by a constant cross-sectional diameter, along all or a portion of the length of the nozzle, or a taper over along all or a portion of its length. The nozzle can be formed integrally with or separately from the retaining member 104. In some embodiments, the nozzle can be selectively attachable and detachable from a dispenser 118 or a tube 120. In some embodiments, the nozzle can form a part of the dispenser 118 and be selectively attachable and detachable from the retaining member 104.

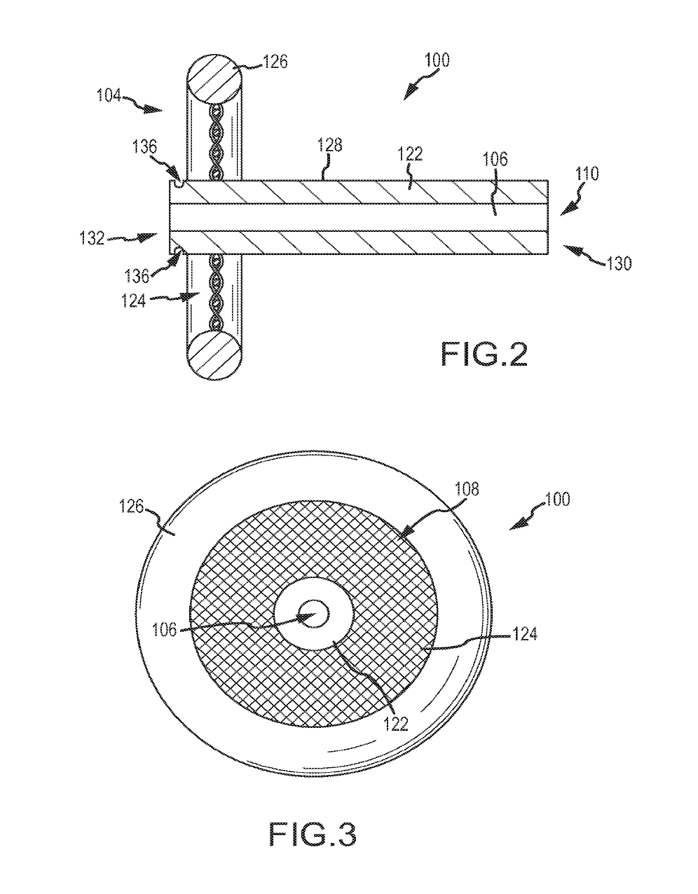

FIGS. 2 and 3 illustrate an embodiment of a delivery device 100 comprising a retaining member 104 and a nozzle 122. As illustrated in FIGS. 2 and 3, the retaining member 104 can comprise a mesh 124. A plurality of outlet passages 108 can extend through the mesh 124. The mesh 124 can be surrounded by a ring 126 as illustrated in FIGS. 2 and 3. The ring 126 can configured to support a radially outer extent of the mesh 124, increase comfort to the ear, or both.

As illustrated in FIGS. 2 and 3, the nozzle 122 can comprise a lumen or delivery passage 106, an outer surface 128, and a distal end 130 and a proximal end 132. The delivery passage 106 can sized to permit movement of the air and particles, e.g. particles of medication or other substance suspended in air, therethrough. The delivery passages 106 and outlet passages 108 can be configured such that a rate of delivery of a stream, spray, or other disbursement of particles to the external auditory canal 102 through the delivery passage 106, and a resistance to flow through the outlet passages 108 do not produce an undesirable increase in localized or general pressure within the external auditory canal.

The nozzle 122 can comprise one or more coupling members 136 for selective coupling and de-coupling with the dispenser 118 or tube 120. As illustrated in the embodiment of FIG. 2, the coupling members 136 can be one or more recesses in the nozzle 122. The coupling members 136 can cooperate with complementary shaped projections of the dispenser 118 or tube 120. In other embodiments, the nozzle 122 can comprise one or more coupling members 136 that comprise projections that cooperate with one or more complementary shaped recesses in the dispenser 118 or tube 120. Selective attachment and detachment of the nozzle 122 and retaining member 104 from the dispenser 118 or tube 120 can permit the nozzle 122 and retaining member 104 to be used and then easily detached from the dispenser 118 or tube 120 for disposal and facilitate attachment of a new nozzle 122 and retaining member 104 to the dispenser 118 or tube 120 for subsequent use, e.g., with a different individual. In some embodiments, the nozzle 122 can be attached to a nozzle of the dispenser 118, such as the tube 120 for example. In some embodiments, the nozzle 122 can be selectively attachable and detachable from the retaining member 104. The coupling members 146 of the dispenser 118 can be configured to cooperate with the coupling members 136 of the body 138 by snap fit, or by other interaction, such as by selective radial contraction and retraction of the coupling members 146.

The retaining member 104 can be attached to the nozzle 122 at a location spaced from the distal end 130, as illustrated in FIG. 2 for example, or at the distal end 130. The retaining member can be spaced a sufficient distance from the distal end 130 of the nozzle 122 to permit the nozzle 122 to extend into the external auditory canal a distance that is sufficient to direct the substance into the extended ear canal, but not so far as to pose a significant risk of harm to the ear.

As illustrated in FIGS. 2 and 3, the retaining member 104 extend laterally from the outer surface 128 of the nozzle, the delivery passage 106, or both. As illustrated in FIG. 3, the retaining member 104 can surround the nozzle 122 in some embodiments. A perimeter of the retaining member 104 can be constantly spaced from the nozzle 122 around the circumference of the retaining member 104, as illustrated in FIGS. 2 and 3. In some embodiments, the retaining member 104 can extend a greater distance from the nozzle 122 at some radial locations than others. The nozzle 122 and the delivery passage 106 can be eccentrically located in the retaining member 104. Although FIGS. 2 and 3 show the retaining number 104 as extending laterally from, but not axially along the nozzle 122 from a connection between the nozzle and the retaining member, the retaining member can extend distally, proximally, or both from the connection location in some embodiments.

The retaining member 104 can be sufficiently large to inhibit insertion of the device 100 into the external auditory canal beyond desired distance. For example, the ring 126 of the device 100 shown in FIGS. 2 and 3 can be sufficiently large to contact the outer ear, and the ring 126 and mesh 124 can resist insertion of the nozzle 122 into the ear when the ring 126 contacts the outer ear.

The size and number of outlet passages 108 in the mesh 124 can be configured such that, considering the rate of delivery of the substance and air, will allow sufficient passage of air through the outlet passages 108 to pose little or no risk of damage to the ear. For example, the size and number of outlet passages 108 can allow a flow rate of air, and possible an amount of the substance, escaping the external auditory canal through the retaining member 104 to be comparable to the rate of delivery of the substance and air to the external auditory canal. In some embodiments, the size and number of outlet passages 108 can be sufficient to avoid rapid buildup of pressure within the external auditory canal upon delivery. In some embodiments, the individual outlet passages 108 have a size that is sufficiently small to inhibit passage of particles of the substance that are delivered to the ear from passing through the outlet passages 108. For example, in some embodiments, the outlet passages 108 can have a characteristic dimension, e.g., a diameter, that is less than a particulate dimension, e.g., an average size of the substance particles to be delivered.

In some embodiments, the mesh 124 illustrated in FIGS. 2 and 3 can be a braid of wires, filaments, ribbons, or other elements. The wires, filaments, ribbons, or other elements can be made of metal, plastic, or other materials, or a combination thereof. In some embodiments, the mesh 124 can be a single, monolithic unit. In some embodiments, the mesh 124 and ring 126 can be integrally formed as a single, monolithic unit. In addition or alternative to the mesh 124, the retaining member 104 can comprise a membrane (not shown) comprising a plurality of outlet passages 108. In some embodiments that comprise a mesh 124 and a membrane, the membrane can extend along the mesh between the nozzle 122 and the ring 126.

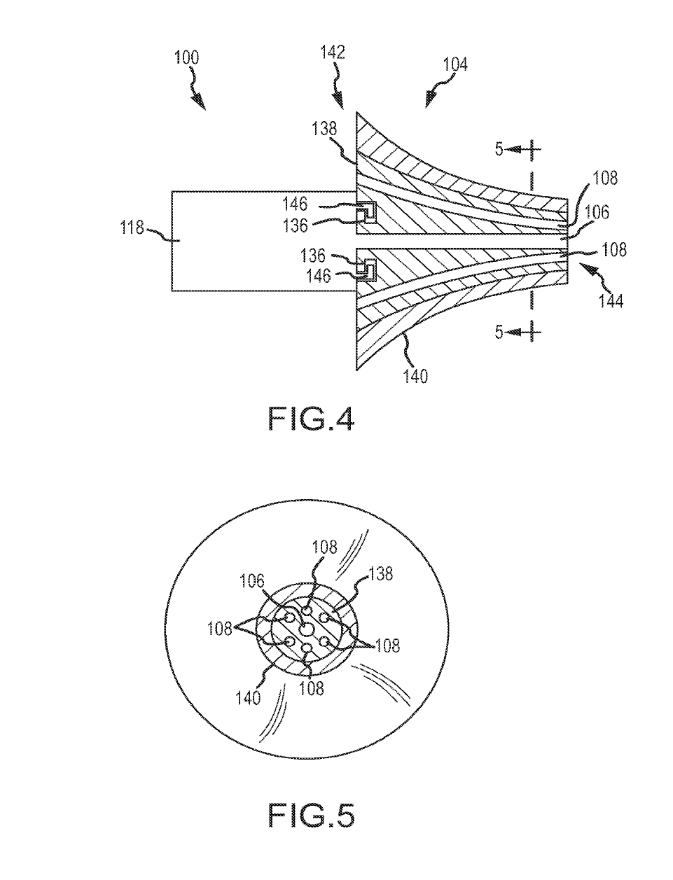

FIGS. 4 and 5 illustrate an embodiment of a device 100 comprising a retaining member 104 and a dispenser 118. The device 100 of FIGS. 4 and 5 is similar to the device 100 of FIGS. 2 and 3 in some respects and different in others. As illustrated in FIGS. 4 and 5, the retaining member 104 can comprise a body 138 and a cover 140. The body 138 can have a plurality of passages extending therethrough including a delivery passage 106 and a plurality of outlet passages 108. Although the embodiment illustrated in FIGS. 4 and 5 has a single delivery passage 106 and six outlet passages 108, other embodiments can have more or fewer passages than those illustrated. The configuration of delivery passage 106 and outlet passage 108 illustrated in FIGS. 4 and 5 is similar that of the delivery and outlet passages discussed above in connection with the embodiment of FIGS. 2 and 3.

As illustrated in FIGS. 4 and 5, the retaining member 104 can taper from a between a proximal end 142 to a distal end 144. The body 138 can be configured of a material sufficiently rigid to resist collapsing of the delivery passage 106 and outlet passages 108 under the forces typically expected upon insertion of the retaining member 104 in the ear. The body 138 can be formed of metal, plastic, or other materials, or combinations thereof. In some embodiments, the body 138 can be formed of material suitable for inexpensively production of disposable retaining members 104. Although the body 138 is illustrated in FIGS. 4 and 5 as a single, monolithic component, the body 138 can comprise a plurality of components in some embodiments.

As shown in FIGS. 4 and 5, the cover 140 can surround a circumference of the body 138 along a length of the body 138 between the proximal end 142 and the distal and 144 of the retaining member 104. The cover 140 can comprise a material that compresses more easily than the body 138 to improve the comfort to the patient upon insertion of the retaining member 104 to the ear by conforming to the shape of a particular individual's ear. In some embodiments, the cover 140 can seal or substantially seal against the outer ear, the external auditory canal, or both.

The body 138, the layer 140, or both can taper between the proximal end 142 and the distal and 144 of the retaining member 104. In embodiments wherein the body 138 and the layer 140 both taper, they can taper in the same direction or opposite directions. Flaring of the retaining member 104 from the distal end 144 to the proximal end 142 can inhibit over insertion of the retaining member into the ear. The distal end of the retaining member 104 can be sufficiently small to be inserted a safe distance within the external auditory canal, or to guide the distal end 144 to a location at the outer-most portion of the external auditory canal.

As illustrated in FIG. 4, the retaining member 104 can comprise one or more coupling members 136 at a proximal end 142. The coupling members 136 can be formed in the body 138 to cooperate with one or more complimentary shaped coupling members 146 of a dispenser 118, as illustrated, or alternatively a tube 120. As discussed above in connection with the embodiment illustrated in FIGS. 2 and 3, the coupling members 136 and 146 can have a variety of configurations.

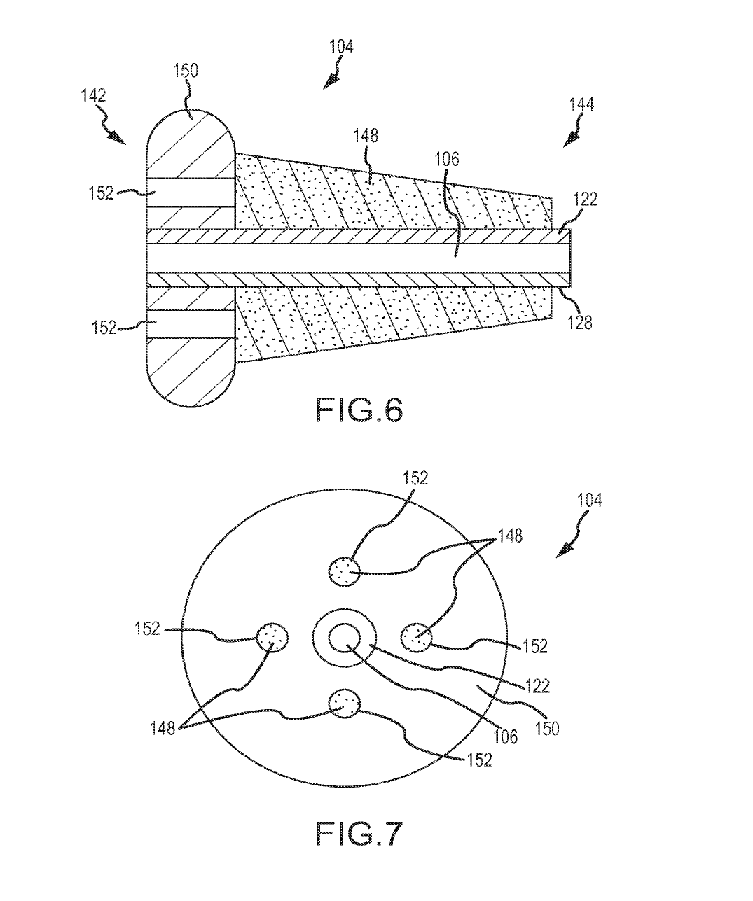

FIGS. 6 and 7 illustrate an embodiment of a retaining member 104 comprising a nozzle 122 and a permeable body 148. The embodiment of FIGS. 6 and 7 is similar to other embodiments disclosed herein in some respects. For example, the nozzle 122 illustrated in FIGS. 6 and 7 comprises a delivery passage 106 extending therethrough. On the other hand, the embodiment of FIGS. 6 and 7 differs from the other embodiments in some respects.

In some embodiments, such as the embodiment illustrated in FIGS. 6 and 7, a retaining member 104 can comprise a permeable body that surrounds the nozzle 122. The permeable body 148 can made of a porous material, such as an a open cell foam, for example, that provides outlet passages that permits air to pass through the permeable body 148. The outlet passages provided by the permeable body 148 can be very small, yet numerous enough to allow sufficient air to pass through it upon delivery of substance and air through the delivery passage 106.

The permeable body 148 can extend between the proximal end 142 and the distal end 144 of the retaining member 104. As illustrated in FIG. 6, the permeable body 148 can extend to a location proximate, yet spaced from, the distal end 144 of the retaining member 104 such that the nozzle 122 extends distally from a distal end of the permeable body 148. However, in some embodiments, the permeable body 148 can extend to or past the distal end of the nozzle 122.

The permeable body 148 can be sized and shaped to engage the external auditory canal, the outer ear, or both. In some embodiments, the permeable body can seal or substantially seal against the ear such that particles of the delivered substance are prevented or inhibited from escaping the ear through space between the permeable body and the ear. The permeable body can be sufficiently resilient to be compressed prior to insertion into the ear to ease insertion, increase comfort to the ear, or both, yet expand rapidly enough to engage the ear and provide outlet passages for the escape of air from the external auditory canal during delivery of the substance and air.

In some embodiments, the retaining member 104 can further comprise a stop member 150, as illustrated in FIGS. 6 and 7, for example. The stop member 150 can be located generally adjacent to the permeable body 148 at a proximal end 142 of the retaining member 104 as shown in FIGS. 6 and 7. In some embodiments, the stop member can be positioned distally of the permeable body. In some embodiments, the stop member can be spaced from the permeable body. The stop member 150 can be configured to prevent or to inhibit insertion of the retaining member 104 into the ear beyond a safe insertion distance. The stop member 150 can comprise one or more openings 152 that facilitate the escape of air from the ear. For example, air can pass through the permeable body 148 and the openings 152 in the stop member 150 to escape the external auditory canal.

The stop member 150 can be attached directly to the nozzle 122 in some embodiments. In other embodiments, the stop member can be attached to another portion of the retaining member 104, such as the permeable body 148 (FIGS. 6 and 7) or the mesh 124 (FIGS. 2 and 3) and spaced from the nozzle 122 (if present). The stop member 150 can be formed as a ring 126 (FIGS. 2 and 3) in some embodiments, for example. In some embodiments, the nozzle 122 and the stop member 150 can be integrally formed.

Although not illustrated in FIGS. 6 and 7, the retaining member 104 can comprise one or more coupling members 136 for attachment to a dispenser 118 or a tube 120, such as described in connection with other embodiments. For example, the nozzle 122, the stop member 150, or both can comprise a coupling member.

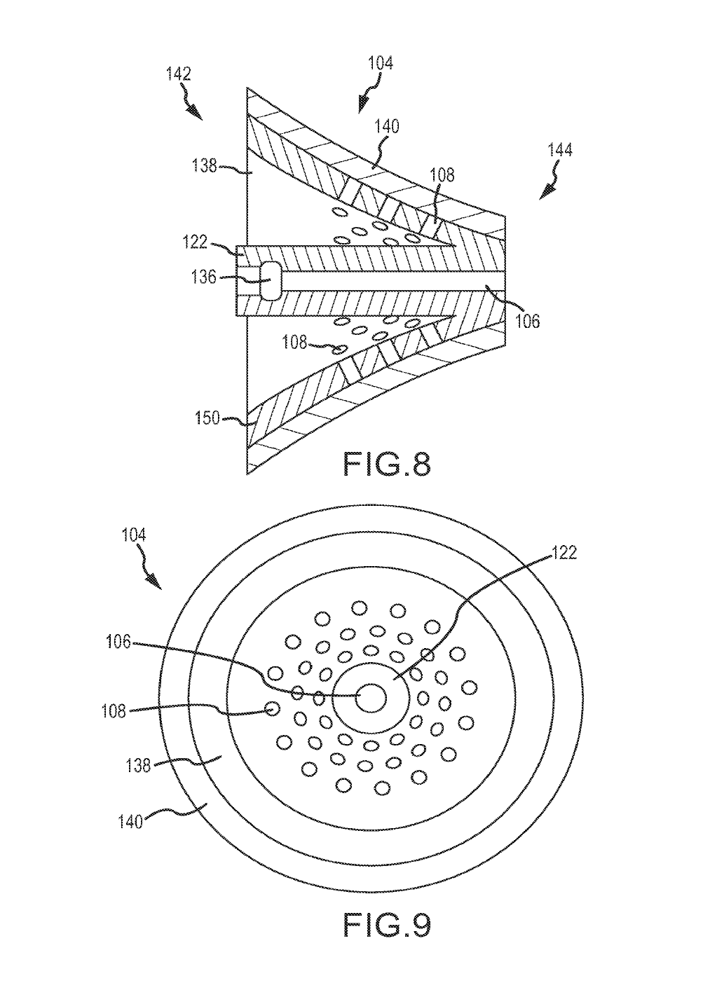

FIGS. 8 and 9 illustrate an embodiment of a retaining member 104 comprising a body 138 and a layer 140. The embodiment illustrated in FIGS. 8 and 9 is similar to the other illustrated exemplifying embodiments in some respects and differs from them in others.

As illustrated in FIGS. 8 and 9, the body 138 can comprise a tube forming a nozzle 122 and a stop 150. The nozzle 122 and the stop 150 can be integrally formed as illustrated in FIG. 8, or can be separately formed. In some embodiments, the stop 150 can be attached to a nozzle of a dispenser 118 for delivery of a substance. The nozzle 122 and the stop 150 can intersect at or near the distal end 144. In some embodiments, the stop 150 can be attached to the nozzle at a location proximally or distally of a distal end of the nozzle.

The stop 150 can flare in the proximal direction as illustrated in FIG. 8. The proximal end of the stop 150 can be sufficiently large to inhibit insertion of the retaining member 104 into the external auditory canal beyond a safe distance. The distal end of the stop member 150 can be sufficiently small to allow insertion of the retaining member 104 within the ear canal a safe distance, or facilitate positioning of the distal end 144 of the retaining member 104 at a proximal end of the external auditory canal without entering the external auditory canal.

In some embodiments, the stop member 150 can be sufficiently rigid to avoid substantial deformation upon insertion of the retaining member 104 to the outer ear, the external auditory canal, or both. In some embodiments, the stop member 150 can be sufficiently flexible to deform upon insertion of the retaining member 104 within the external auditory canal such that the stop member at least partially conforms to the shape of a particular outer ear, external auditory canal, or both. The stop 150 can comprise a plurality of outlet passages 108 to permit air, and possibly particles of the delivered substance, to escape during delivery.

The layer 140 shown in FIGS. 8 and 9 can be similar to the layer 140 illustrated in FIGS. 4 and 5. The layers 140 described in connection with FIGS. 4-5 and 8-9 can be similar to the permeable body 148 described in connection with FIG. 148. The layer 140 can be formed of a permeable material providing passages to allow the flow of air, and possibly particles of the delivered substance, through the layer 140. Although the layer 140 is illustrated as having a generally constant thickness between the proximal end 142 and the distal end 144, in some embodiments, the layer can vary in thickness between the proximal end 142 and the distal end 144. For example, the proximal end 142 can be thicker than the distal end 144. In some embodiments, the layer 140 can be thinner near the proximal end 142 than near the distal end 144. The layer 140 can be compressible to conform to the shape of the ear while maintaining sufficient permeability to allow air, and possibly particles of the delivered substance, to escape the ear. The ability of the retaining member 104 to deform while maintaining sufficient permeability during insertion of the retaining member 104 to the ear and delivery of the substance can be desirable so that a retaining member 104 can conform to the unique ear shapes of a variety of individuals.

Similar to other exemplifying embodiments, the embodiment illustrated in FIGS. 8 and 9 comprises a coupling member 136 for attachment to a dispenser 118, a tube 120 or both. Although FIG. 8 shows the coupling member 136 as a single groove extending circumferentially within the delivery passage 106, the coupling member 136 can take other configurations such as those described or shown in connection with other embodiments.

The apparatuses disclosed herein can be used to deliver a medication or other substance to an external auditory canal. For example, the retaining member can be positioned at or near an opening of the external auditory canal. In some embodiments, a portion or all of the retaining member 104 can be inserted into an outer portion of the external auditory canal. In embodiments wherein the retaining member 104 comprises a compressible material, e.g., the layer 140 or permeable body 148, the compressible material can be compressed prior to the insertion of the retaining member 104 to the ear. In some embodiments, the retaining member 104 can be positioned against the outer ear such that passage 106 directs the air and substance into the external auditory canal 102 (FIG. 1). For example, the passage 106 can be aligned with the external auditory canal.

With the retaining member 104 placed at or within the eternal ear canal, the dispenser 118 can be actuated to mix a dose, e.g., a metered dose, of a substance to be delivered, e.g., a medication. Actuation of the dispenser can de-aggregate a dose of the substance from a large quantity, as indicated above. Particles of the substance and air can be urged through a tube 120 (if present), and the retaining member 104 into the ear. As the substance is introduced to the external auditory canal, the retaining member 104 can inhibit egress of at least some of the substance from the external auditory canal. For example, one or more outlet passages, such as the outlet passages 108 or passages through the layer 140 or the permeable body 148, can inhibit passage of the substance while permitting passage of the air to escape the external auditory canal. In some embodiments, the retaining member 104 can expand, conform, or both to the ear. In some embodiments, the retaining member 104 can seal or substantially seal against the outer ear, the external auditory canal or both.

Although embodiments have been primarily described herein with reference to the external auditory canal, the disclosed embodiments can be configured to deliver substances to other locations, e.g., the nasal cavity or oral cavity, in additional or alternative to the external auditory canal. For example, the retaining member 104 can be configured to engage a nostril such that an exterior surface of the retaining member substantially sealingly engages an interior surface of the nostril when inserted therein.

In some embodiments, the retaining member 104 is configured to engage independently each of (i) an external auditory canal and (ii) a nostril such that the same delivery device can be used to deliver substances to both the ear and nose. For example, an exterior surface of the retaining member 104 can be configured such that a portion of the exterior surface substantially sealingly engages an interior surface of the nostril when inserted therein, and the same portion or a different portion of the exterior surface substantially sealingly engages a surface of the ear in or near the external auditory canal when inserted therein.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A device for delivery of particulates to an external auditory canal, the device comprising:
a reservoir configured to contain a substance to be delivered to the external auditory canal;
an aerosolizing mechanism configured to suspend a dose of the substance, as particulates, in a gas;
a nozzle comprising (i) a lumen sized to permit movement of the suspended particulates therethrough, (ii) an outer surface, and (iii) a distal end;
a retaining member configured to be attached to the nozzle proximate the distal end of the nozzle, projecting laterally from the lumen of the nozzle, shaped to engage an external portion of the ear, and comprising a passage therethrough configured to allow passage of air and to inhibit passage of at least some of the particulates.

2. The device of claim 1, wherein the particulates are not liquid.

3. The device of claim 1, wherein the retaining member completely surrounds a circumference of the nozzle.

4. The device of claim 1, further configured such that a volumetric flow rate through the nozzle and a volumetric flow rate through the retaining member are substantially the same during delivery.

5. The device of claim 1, wherein at least some of the one or more passages of the retaining device have a characterizing dimension that is smaller than a particulate dimension.

6. The device of claim 1, wherein the one or more passage of the retaining device are formed by an open-cell foam.

7. The device of claim 1, wherein the one or more passage of the retaining device are formed by a mesh.

8. The device of claim 1, wherein the retaining member further comprises a resilient portion configured to conform to a shape of the external ear and wherein the one or more passages of the retaining member are surrounded by one or more rigid walls.

9. The device of claim 1, wherein the retaining member impedes advancement of the nozzle into the ear.

10. The device of claim 9, wherein the retaining member has a portion with an outer dimension that is significantly larger than an opening to the external auditory canal.

11. The device of claim 10, wherein the portion of the retaining member is at a distal end of the retaining member.

12. The device of claim 1, wherein the retaining member is configured to be selectively attached and removed from the nozzle.

13. The device of claim 1, wherein the retaining member comprises a plurality of passages therethrough configured to allow passage of air and to inhibit passage of at least some of the particulates.

14. The device of claim 1, wherein the substance to be delivered comprises one or more ingredients selected from the group consisting of antibiotics, antibacterial agents, bacteriocides, bacteriostatic agents, antifungals, antivirals, analgesics, anesthetics, anti-inflammatories, nonsteroidal anti-inflammatory drugs, immunomodulators, tacrolimus, pimecrolimus, cerumenolytics, Debrox, carbamide peroxide, glycerine, sodium bicarbonate, Cerumol, Cerumenex, Exterol, and Docusate.

15. A method for delivering particulate material to an external auditory canal, comprising:
aerosolizing a dose of a substance; delivering the substance to the external auditory canal through a nozzle; and
inhibiting egress of at least some of the substance from the external auditory canal between the nozzle and a wall of the external auditory canal.

16. The method of claim 15, wherein said inhibiting comprises reducing an amount of cross-sectional area between the nozzle and the external auditory canal through which air and the substance can escape the external auditory canal.

17. The method of claim 16, wherein said inhibiting further comprises spanning an opening of the external auditory canal with a retaining member attached to the nozzle proximate the distal end of the nozzle and comprising one or more passages therethrough configured to allow passage of air and to inhibit passage of at least some of the particulates.

18. The method of claim 16, wherein the substance is delivered to treat an external auditory canal infection.

19. The method of claim 16, wherein the substance is delivered to treat an abscess.

20. The method of claim 15, wherein the substance comprises one or more ingredients selected from the group consisting of antibiotics, antibacterial agents, bacteriocides, bacteriostatic agents, antifungals, antivirals, analgesics, anesthetics, anti-inflammatories, nonsteroidal anti-inflammatory drugs, immunomodulators, tacrolimus, pimecrolimus, cerumenolytics, Debrox, carbamide peroxide, glycerine, sodium bicarbonate, Cerumol, Cerumenex, Exterol, and Docusate.
